(19) United States
(12) Patent Application Publication
(10) Pub. No.: US 2010/0029590 A1
(43) Pub. Date: Feb. 4, 2010

(54) METHODS FOR TREATING OR PREVENTING HEMORRHAGIC CYSTITIS USING A GLYCEROPHOSPHATE SALT

(75) Inventor: Alan E. Kligerman, Egg Harbor Township, NJ (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: PRELIEF INC., Egg Harbor Township, NJ (US)

(21) Appl. No.: 12/526,959

(22) PCT Filed: Feb. 13, 2008
(86) PCT No.: PCT/US08/53822

§ 371 Date: Aug. 13, 2009

Related U.S. Application Data
(60) Provisional application No. 60/889,770, filed on Feb. 14, 2007.

(51) Int. Cl. A61K 31/66

(52) U.S. Cl. 514/75

(57) ABSTRACT

Glycerophosphate salts have been found to mitigate the syndromes or conditions of discomfort associated with hem-orrhagic cystitis. Therefore, methods are provided for treating or preventing the syndromes or conditions of discomfort associated with hemorrhagic cystitis using a glycerophosphate salt, hi particular, methods are provided for treating or preventing the syndromes or conditions of discomfort associated with hemorrhagic cystitis using calcium glycerophosphate (CGP).

### BACKGROUND OF THE INVENTION

Cystitis is the inflammation of the bladder. There are several types of cystitis, including, but not limited to, bacterial cystitis caused by coliform bacteria, interstitial cystitis of unknown cause characterized by urinary frequency (as often as every 10 minutes), urgency, pressure and/or pain in the bladder and/or pelvic cavity, and hemorrhagic cystitis, which is diffuse inflammation of the bladder leading to hemorrhage. Hemorrhagic cystitis is seen most often in cancer patients as a complication of therapy. Causes of hemorrhagic cystitis include, but are not limited to, chemotherapy (e.g., cyclophosphamide therapy), radiation, or viral infection (e.g., with the BK virus in bone marrow transplant patients). For example, hemorrhagic cystitis can occur about 1 month to 10 years after pelvic radiation therapy with moderate to severe persistent rates of hematuria as 3% to 5% after radiotherapy for pelvic malignancies. Current treatment modalities for hemorrhagic cystitis include oral and intravenous agents, intravesical therapy and selective embolization of the hypogastric arteries. In addition, hyperbaric oxygen therapy has been shown to be effective in patients in whom other forms of management have failed. Corman et al., J. Urol., 169:2200-2 (2003).

In addition to extreme symptoms of urgency, pressure and/or pain in the bladder and/or pelvic cavity similar to those of interstitial cystitis, possible complications of hemorrhagic cystitis include, for example, chronic or recurrent urinary tract infection (UTI), complicated UTI (pyelonephritis), and acute renal failure. Therefore, there is a need to develop novel therapy for the effective treatment or management of hemorrhagic cystitis.

### BRIEF SUMMARY OF THE INVENTION

It is now discovered that glycerophosphate salts can be used to treat or inhibit the onset of hemorrhagic cystitis.

In one general aspect, an embodiment of the present invention comprises a method of treating a syndrome or a condition of discomfort associated with hemorrhagic cystitis in a subject. The method comprises administering to the subject a therapeutically effective amount of a glycerophosphate salt, to interdict, prevent, palliate, or alleviate the syndrome or the condition of discomfort of the subject associated with the hemorrhagic cystitis.

In another general aspect, an embodiment of the present invention comprises a method of preventing or inhibiting the onset of a syndrome or a condition of discomfort associated with hemorrhagic cystitis in a subject. The method comprises administering to the subject a prophylatically effective amount of a glycerophosphate salt, to interdict, prevent, palliate, or delay the onset of the syndrome or the condition of discomfort of the subject associated with the hemorrhagic cystitis.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawing. For the purpose of illustrating the invention, there is shown in the drawing embodiments of the invention. It should be understood, however, that the invention is not limited to the precise arrangements and instrumentalities shown.

In the drawing:

FIG. 1 shows the average number of micturinations per night as a function of time in a patient subject to brachytherapy treatment (Jan. 14, 2002), oral administration of Prelief® (starting 1/2003), and the insertion and removal of a stent.

### DETAILED DESCRIPTION OF THE INVENTION

Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. In this application, certain terms are used frequently, which shall have the meanings as set in the specification. It must be noted that as used herein and in the appended claims, the singular forms “a,” “an,” and “the” include plural reference unless the context clearly dictates otherwise.

Embodiments of the present invention comprise methods of treating or preventing a syndrome or a condition of discomfort associated with hemorrhagic cystitis in a subject comprising administering to the subject an effective amount of a glycerophosphate salt, thereby interdicting, preventing, delaying the onset of, palliating, or alleviating, the syndrome or the condition of discomfort of the subject associated with the hemorrhagic cystitis.

As used herein, the term “hemorrhagic cystitis” refers to bladder inflammation as the result of a chemical or other traumatic insult to the bladder. A syndrome or a condition of discomfort associated with hemorrhagic cystitis can be, for example, discomfort and an urge to urinate often, pressure and/or pain in the bladder and/or pelvic cavity, the presence of blood cells in urine visible to bare eyes or under a microscope, or epithelial proliferations that appear similar to invasive urothelial carcinomas.

As used herein, the term “pelvic cavity” shall mean not only the physical boundaries of a body cavity that is bounded by the bones of the pelvis and that primarily contains reproductive organs, the urinary bladder and the rectum, in the strict medical terminology, but also all that/those part(s) of the body which are neurologically and sympathetically connected to and/or responsive to (a) events which are generated within the body cavity bounded by the bones of the pelvis and are resonated to other locations in the body, and (b) events which are generated elsewhere in the body and are resonated to within the body cavity bounded by the bones of the pelvis. Such events are common, given the peculiar neural connections which exist within the body that tie the pelvic areas to regions which may not normally be considered strictly “pelvic”. For example: the external genitalia, urinary bladder and intestines have a neural commonalty in their pre-synaptic neutral connections which proceed separately from the coccyl region of the spine, tie together as a neural bundle, then separate again, to proceed to each of the three entities stated above. The post-synaptic nerves from each entity of external genitalia, urinary bladder and intestines, then depart each entity separately, tie together once more as a neural bundle, then separate again to proceed back to the spinal column at the lumbar region. Therefore, the brain may confuse in interpreting where a particular pain signal in fact arises. Persons believing they experience pain in, e.g., the urethra, may in fact have no authentic pain source in the urethra, but have authentic pain source in the bladder or prostate instead. This may occur due to a false or ambiguous message delivered by the neural bundle to the brain. It is shown in medical literature that while about 15% of the general U.S. population experiences irritable bowel syndrome (IBS), approximately 75% of persons with interstitial cystitis (IC) report IBS symptoms, indicating that the problem of bladder, therefore pelvic cavity, occurrence, e.g., IC, appears to have an unmistakeable influence on the intestines, which are not strictly within the pelvic cavity, lying within the more commonly referenced “bowel.”

As used herein, “pain” refers to an unpleasant sensation associated with actual or potential tissue damage and mediated by specific nerve fibers to the brain where its conscious appreciation may be modified by various factors. The unpleasant sensation of pain ranges in intensity from slight through severe to indescribable. Pain is experienced as having qualities such as stinging, sharp, throbbing, dull, nauseating, burning and shooting.

In one embodiment of the invention, the hemorrhagic cystitis is caused by a chemotherapy, which is thus called “chemotherapy cystitis”. As used herein, the term “chemotherapy” refers to a treatment of a disease by means of a chemical that has a toxic effect upon the disease-producing cells, such as the disease-producing microorganisms (yeasts, fungi, bacteria or viruses) or the cancerous cells. In particular embodiments, a “chemotherapy” refers to a treatment of a neoplastic disease, such as a tumor, which includes a cancer. In some embodiments, a chemotherapy is used to cure some types of cancer. In other embodiments, a chemotherapy is used to slow the growth of cancerous cells or to keep the cancer from spreading to other parts of the body. In addition, when a cancer has been removed by surgery, a chemotherapy can be used to keep the cancer from coming back as an adjuvant therapy. Chemotherapies also can ease the symptoms of cancer, helping some patients to have a better quality of life. Methods of the invention can be used to treat or prevent a syndrome or a condition of discomfort associated with chemotherapy cystitis resulting from or associated with any chemotherapy.

In another embodiment of the invention, the hemorrhagic cystitis is caused by a radiation therapy, which is thus also called “radiation cystitis”. As used herein, the term “radiation therapy”, also called “radiotherapy”, “brachytherapy”, “x-ray therapy”, “cobalt therapy”, “electron beam therapy” or “irradiation”, refers to a treatment of a disease using penetrating waves or particles such as x-rays, gamma rays, proton rays, or neutron rays to destroy or damage the disease-producing cells, such as the disease-producing microorganisms (yeasts, fungi, bacteria or viruses) or the cancerous cells. In preferred embodiments, a “radiation therapy” refers to a treatment of a neoplastic disease, such as a tumor which includes a cancer. A radiation therapy is a common form of cancer therapy. It is used in more than half of all cancer cases. A radiation therapy can be used, for example, alone to kill cancer, before surgery to shrink a tumor and make it easier to remove, during surgery to kill cancerous cells that may remain in surrounding tissue after the surgery (called intraoperative radiation), after surgery to kill cancerous cells remaining in the body, to shrink an inoperable tumor in order to reduce pain and improve quality of life, or in combination with chemotherapy.

A radiation therapy can be given externally and/or internally. In external radiation therapy, a machine directs the high-energy rays at the cancer and a small margin of normal tissue surrounding it. When internal radiation therapy is used, the radiation source is placed inside the body. This internal method of radiation treatment is called brachytherapy or implant therapy. The source of the radiation (such as radioactive iodine) sealed in a small holder is called an implant. Implants may be thin wires, plastic tubes (catheters), capsules, or seeds. An implant may be placed directly into a tumor or inserted into a body cavity. Sometimes, after a tumor has been removed by surgery, the implant is placed in the ‘tumor bed’ (the area from which the tumor was removed) to kill any tumor cells that may remain. Methods of the invention can be used to treat or prevent a syndrome or a condition of discomfort associated with radiation cystitis resulting from or associated with any radiation therapy. In an embodiment of the present invention, methods of the invention can be used to treat or prevent radiation cystitis resulting from or associated with pelvic radiation therapy, which is used, for example, to treat endometrial tumor, cervical tumor, rectal tumor, prostate tumor.

Methods of the present invention can also be used to treat or prevent damage to cells, particularly epithelial cells, and tissues of the bladder resulting from chemotherapy or radiation therapy used to treat any genital tumors, such as penile tumors and ovarian tumors. It is conceivable that methods of the present invention can also be used to treat or prevent damage to cells, particularly epithelial cells, or tissues of the bladder, resulting from chemotherapy or radiation therapy used to treat other types of diseases, such as tumors in breast, esophagus, brain, jaw, bone, soft tissue, etc. In one embodiment of the present invention, the present method can be used to prevent or treat mucositis, i.e., inflammation of the mucous membranes, in the bladder or pelvic cavity. The mucositis usually occurs as an adverse effect of chemotherapy and/or radiation therapy for cancer.

In yet another embodiment, methods of the invention can be used to treat or prevent a syndrome or a condition of discomfort associated with hemorrhagic cystitis resulting from or caused by viral infection. For example, hemorrhagic cystitis caused by adenoviral infection is a known complication of allogenic bone marrow transplant (BMT). Methods of the invention can be used to prevent or treat such BMT complication.

Methods of the invention can also be used to treat or prevent a syndrome or a condition of discomfort associated with hemorrhagic cystitis resulting from or caused by a combination of a chemotherapy, a radiation therapy, and/or a viral infection with one or more other therapies, such as a surgery.

As used herein, the term “subject” refers to an animal, preferably a mammal, most preferably a human, who has been the object of treatment, observation or experiment. Examples of a subject can be a human, a livestock animal (beef and dairy cattle, sheep, poultry, etc.), a companion animal (dog, cat, horse, etc), or a race animal, e.g., a race horse.

As used herein the term “treatment”, “treat” or “therapy” refers to the prevention of deterioration of a disease, disorder or condition when a patient contracts such a disease, disorder or condition, preferably, at least maintenance of the status quo, and more preferably, alleviation, still more preferably, resolution of the disease, disorder or condition.

As used herein the term “prophylaxis”, “prevent” or “prevention” refers to, when referring to a disease, disorder or condition, a type of treatment conducted before such a disease, disorder or condition occurs such that the disease, disorder or condition will not occur, will be delayed to occur, or will occur but will deteriorate to a less degree.

As used herein, the term “treat” or “prevent” in the broadest sense, with respect to a disease, disorder or condition, refers to any medical act thereto, and include any act for diagnosis, therapy, prevention, prognosis and the like.

When used for treating or preventing a syndrome or a condition of discomfort associated with hemorrhagic cystitis, glycerophosphate salt can be used as a reliever which is used after the onset of hemorrhagic cystitis. Glycerophosphate salt can also be used as a controller which is used for long-term control to prevent the occurrence of hemorrhagic cystitis. Those skilled in the art will be able to use an effective amount of glycerophosphate salt for either therapy or prevention of hemorrhagic cystitis.

The term “effective amount” as used herein, means that amount of a glycerophosphate salt that elicits the biological or medicinal response in a tissue system of a subject, or in a subject, that is being sought by a researcher, veterinarian, medical doctor or other clinician, which includes interdicting, preventing, palliating, or alleviating a syndrome or a condition of discomfort of the subject associated with the hemorrhagic cystitis being treated. The administration of an effective amount of glycerophosphate salt to a subject results in a clinically observable beneficial effect. The clinically observable beneficial effect can be a situation that an observable syndrome or condition of discomfort associated with hemorrhagic cystitis is prevented from further development or aggravation or develop to a lesser degree, than without administration of the composition of the present invention. The clinically observable beneficial effect can also be a situation that a syndrome or a condition of discomfort associated with hemorrhagic cystitis is prevented from occurring or subsequently occurs to a lesser degree than without administration of the composition of the present invention, when the composition is administered to a subject before the hemorrhagic cystitis is observable. The effective amount can be a therapeutically effective amount or a prophylactically effective amount.

Methods are known in the art for determining therapeutically and prophylactically effective doses of glycerophosphate salt according to the present disclosure. A useful assay for confirming an effective amount (e.g., a therapeutically effective amount) for a predetermined application is to measure the degree of recovery from a target disease. An amount actually administered depends on an individual to be treated. The amount is preferably optimized so as to obtain a desired effect without significant side effects. The determination of a prophylactically or therapeutically effective amount is within the ability of those skilled in the art in view of the present disclosure. A prophylactically or therapeutically effective amount of any compound can be estimated using either a cell culture assay or any appropriate animal model. The animal model is used to achieve a desired concentration range and an administration route. Thereafter, such information can be used to determine a dose and route useful for administration into humans.

The therapeutic effect and toxicity of a compound may be determined by standard pharmaceutical procedures in cell cultures or experimental animals (e.g., ED₅₀, a dose therapeutically effective for 50% of a population; and LD₅₀, a dose lethal to 50% of a population). The dose ratio between therapeutic and toxic effects is a therapeutic index, and it can be expressed as the ratio of ED₅₀/LD₅₀. Pharmaceutical compositions which exhibit high therapeutic indices are preferable. The data obtained from cell culture assays and animal studies can be used for formulating a dosage range for use in humans. The dosage of such compounds lies preferably within a range of circulating concentrations that include the ED₅₀, with little or no toxicity. Such a dosage may vary within this range depending upon the dosage form employed, the susceptibility of a patient, and the route of administration. Guidance for specific doses and delivery methods is provided in publications known in the art. Procedures can be performed to evaluate the effect of the administration of a glycerophosphate salt to a subject, thus allowing a skilled artisan to determine the therapeutically effective amount of a glycerophosphate salt to be administered to the subject. For example, changes in the expression level of nuclear matrix protein 22 (NMP22) or anti-proliferative factor (APF), imaging techniques, and/or urinary cytology can be used to determine severity and to assess complications of hemorrhagic cystitis.

The exact dose is chosen by an individual physician in view of the condition of a patient to be treated. Doses and administration are adjusted to provide a sufficient level of the active portion, or to attain a desired effect. Preferably, a therapeutically effective amount of a glycerophosphate salt will reduce a syndrome or a condition of discomfort of the subject associated with the hemorrhagic cystitis under treatment by at least about 20%, for example, by at least about 30%, about 40%, about 50%, about 60%, about 70%, about 80%, about 90%, or about 100%. Preferably, a prophylactically effective amount of a glycerophosphate salt will reduce a syndrome or a condition of discomfort of the subject associated with the hemorrhagic cystitis to be prevented, or the probability of its onset, by at least about 20%, for example, by at least about 30%, about 40%, about 50%, about 60%, about 70%, about 80%, about 90%, or about 100%.

The effective amount of a glycerophosphate salt per administration can be, for example, about 0.1 gram to about 3.0 grams, and preferably about 0.3 gram to about 1.0 gram. The preferred daily dosage of a glycerophosphate salt can be, for example, about 0.6 gram to about 18 grams, more preferably about 1.8 grams to about 6 grams. However, the number of doses per day and the quantity of a glycerophosphate salt which may be administered to a subject can be virtually unlimited.

As used herein, the term “glycerophosphate salt” refers to a chemical compound that is derived from glycerophosphate, in which one or more of the hydrogens of the phosphate group of glycerophosphate are replaced by a basic radical, in particular embodiments by a metal ion. As used herein, the term “glycerophosphate” refers to an anion of a phosphoric ester of glycerol, in which a carbon atom of glycerol bonds to an oxygen atom in the phosphate group of phosphoric acid. A glycerophosphate salt can be a chiral molecule, i.e., it can exist in two forms that are nonsuperimposable mirror images. It is intended that the present invention includes within its scope the stereochemically pure isomeric forms of a glycerophosphate salt and/or their racemates.

In particular embodiments, methods of the invention utilize one or more glycerophosphate salts selected from the group consisting of calcium glycerophosphate (CGP), magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, and glycerophosphate strontium.

In a preferred embodiment, methods of the invention utilize calcium glycerophosphate. As used herein, the term “calcium glycerophosphate” or “CGP”, also known as “glycerophosphate calcium,” refers to a chemical compound having a molecular formula of C₃H₇CaO₆P in its anhydrous form. “CGP” can also exist as a hydrate, including the monohydrate and the dihydrate. Examples of calcium glycerophosphate include, but are not limited to, any one, or any combination of two or more of the three isomers of CGP, namely P-glycerophosphoric acid calcium salt ((HOCH₂)₂CHOPO₃Ca) and D(+) and L(−)-α-glycerophosphoric acid calcium salt (HOCH₂CH(OH)CH₂OPO₃Ca).

Calcium glycerophosphate available from various commercial sources can be used in the present invention. In one embodiment, Prelief®, a dietary supplement for use in reducing the impact of acid in foods and beverages that is available from AkPharma Inc. (Pleasantville, N.J. 08232), in either tablet or powder form, can be used in the present invention for orally administering the calcium glycerophosphate to the subject. Other commercially available CGP also includes CGP available from Astha Laboratories Pvt, Ltd, B-4, Industrial Estate, Sanathnagar, Hyderabad-18, India, and Seppic Inc., 30 Two Bridges Road, Fairfield, N.J.

In particular embodiments, one or more glycerophosphate salts are combined with an acceptable carrier in a composition for administration to a subject. The composition can contain about 0.1 mg to about 3000 mg of a glycerophosphate salt, and can be constituted into any form suitable for the mode of administration selected. Carriers include excipients, including, but not limited to, binders, suspending agents, lubricants, flavorants, sweeteners, preservatives, dyes, and coatings.

To prepare the compositions, one or more glycerophosphate salts, as the active ingredient, are intimately admixed with a carrier according to conventional pharmaceutical compounding techniques, which carrier may take a wide variety of forms depending on the form of preparation desired for administration. In preparing the compositions in oral dosage form, any of the usual pharmaceutical media may be employed. For liquid oral preparations, for example, suspensions, elixirs and solutions, suitable carriers and additives include water, glycols, oils, alcohols, flavoring agents, preservatives, coloring agents and the like; for solid oral preparations, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The compositions herein can contain, per dosage unit, e.g., tablet, capsule, powder, injection, teaspoonful and the like, an amount of the active ingredient necessary to deliver an effective amount as described above.

A glycerophosphate salt can be administered to a subject by one or more of the following methods: orally, via injection or infusion, or internal or external topically. Preferably, a glycerophosphate salt is administered orally. It can be administered with foods, beverages or additional one or more other drugs. It can be orally administered in solid forms, such as pills, tablets, caplets, capsules (each including immediate release, timed release and sustained release formulations), granules, and powders, and/or in liquid forms, such as solutions, syrups, elixirs, emulsions, and suspensions.

A glycerophosphate salt can also be administered by injection or infusion into a vein (intravenous, IV), a muscle (intramuscular, IM), or under the skin (subcutaneous, SC). It can be injected or infused in the form of sterile solutions, emulsions or suspensions. A glycerophosphate salt administered by IM is absorbed into the blood more slowly than IV, thus may have longer lasting effect than IV. A glycerophosphate salt can also be given by a catheter or port permanently inserted into a central vein or body cavity. A port is a small reservoir or container that is placed in a vein or under the skin in the area where the drug will be given. These methods eliminate the need for repeated injections. Catheters and ports can also be placed in bladder or pelvic cavity for intravesicular instillation use.

A glycerophosphate salt can also be given external topically as a cream or ointment applied directly to the surface areas exposed to or affected by the chemotherapy or radiation therapy. Also, a glycerophosphate salt can be administered via transdermal skin patches to provide continuous dosage regimen. A glycerophosphate salt can also be applied internal topically into the rectum, vagina, urine tract, and/or colon, including, but not limited to, the mucous membranes adversely affected by chemotherapy or radiotherapy, by a procedure such as enema, lavage or gavage.

How often and how long a glycerophosphate salt is administered to a subject depends on factors such as the type of treatment or prevention, how the subject responds to the glycerophosphate salt, factors associated with the subject, e.g., age, weight, diet, health, ability to tolerate the glycerophosphate salt, and the types of glycerophosphate salt used. A glycerophosphate salt can be administered on a regimen of one to multiple times per day. Alternatively, a glycerophosphate salt can be formulated in an extended release form suitable for once-daily, once-weekly or once-monthly administration. Methods are known to those skilled in the art to manufacture the extended release dosage form. Preferably, a glycerophosphate salt is administered to the subject at intervals during the day, such as at breakfast, lunch, dinner, and upon retiring. A glycerophosphate salt can be given in combination with one or more other drugs. They can be administered together or one following the other.

In methods of inhibiting the onset of hemorrhagic cystitis of the present invention, a prophylatically effective amount of a glycerophosphate salt can be administered prior to, together with, or after the therapy that causes or results in the hemorrhagic cystitis. For example, prophylatically effective amount of a glycerophosphate salt can be administered to a subject prior to administering to the subject a radiation therapy, a chemotherapy, or a bone marrow transplant to inhibit the onset of a radiation cystitis, a chemotherapy cystitis, or a hemorrhagic cystitis caused by viral infection, respectively. Because it may take months or years after the administration of the radiation or chemotherapy for hemorrhagic cystitis to develop, a prophylatically effective amount of a glycerophosphate salt can also be administered together with or even after the administration of the therapy.

This invention will be better understood by reference to the non-limiting examples that follow, but those skilled in the art will readily appreciate that the examples are only illustrative of the invention as described more fully in the claims which follow thereafter.

### Example

### Prelief® Reduced Symptoms of Hemorrhagic Cystitis

This example depicts a method of using calcium glycerophosphate (CGP) in treating radiation cystitis in a clinical setting. Methods similar to that exemplified herein, however, is equally applicable to using any glycerophosphate salt in treating or preventing any type of hemorrhagic cystitis.

Six male patients suffering from radiation cystitis were observed in a clinical setting. All six patients, aging from 51 to 74 year old at the time, were under prostatic brachytherapy treatment. Each patient showed symptoms of radiation cystitis, such as increased frequency of urination and the experience of pain in bladder. Two Prelief® tablets, each containing about 0.335 gram CGP, were orally administered to each patient three times a day starting at about 0 to 8 months after the brachytherapy. All six patients were observed for 7+ months. Some patients were also treated with another therapy after the radiation, such as by a stent, or with the administration of AINES, Trospium Chloride, or Tamsulosina. On his post brachytherapy visitations, each patient was asked, among other things, the average number of micturitions per night.

Reduced symptoms of radiation cystitis were reported by each of the six patients after the oral administration of CGP. As summarized in Table 1, frequency of urination was generally reduced from about 8-11 times per night to about 4.5-7 times per night after oral administration of CGP for about three months, dropping further to 3-7 times per night for the succeeding seven months. This is in comparison to up to 12 times per night expected micturations in severe cases of this type when patients are given regular medications. The frequency of urination was further reduced by continuing oral administration of CGP. In addition, reduced pain in bladder was also reported by the patients (data not shown). On some occasions, when patients went off Prelief® therapy, their symptoms of radiation cystitis worsened, and upon resumption of regular Prelief® dosing their symptoms again were reduced.

TABLE 1

Average No. of Micturitions/Night After
Oral Administration of Prelief ®

| Patient | 0 month | 3 months | 7 months | 12 months |
|---|---|---|---|---|
| 1* | 11 | 7 | 7 | 7 |
| 2 | 9.5 | 5 | 4 | no observation |
| 3 | 10 | 5 | 3.5 | 3.2 |
| 4* | 9 | 6.5 | 4.7 | 5.5 |
| 5* | 8 | 4.5 | 3 | 5.5 |
| 6 | 9 | 4.5 | 3.5 | no observation |

*noncompliance in regular dosage of Prelief ® was reported.

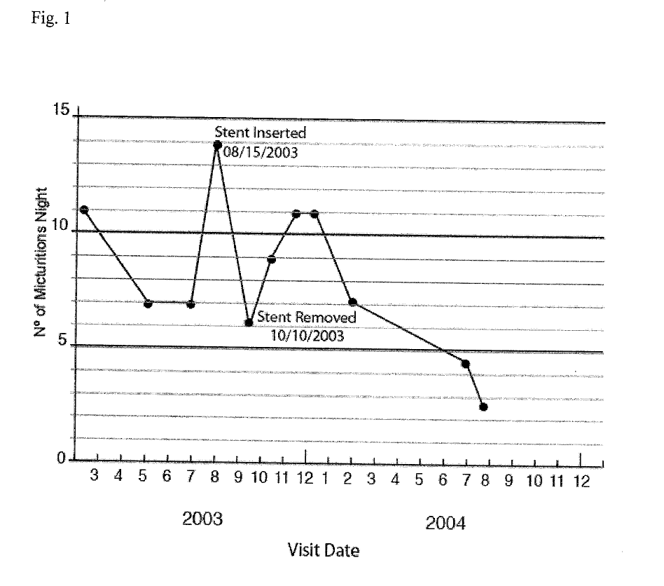

As illustrated in FIG. 1, patient #1, a 66 year old man, was subject to a prostatic brachytherapy treatment on Nov. 14, 2002. The patient showed symptoms of radiation cystitis, e.g., urinating about 11 times per night at about 3 months following the brachytherapy treatment. The patient reported reduced symptoms of radiation cystitis after the oral administration of CGP, e.g., the frequency of urination was reduced to about 7 times per night after oral administration of CGP for about 5 months. Following the insertion and the removal of a stent, the patient showed increased symptoms of hemorrhagic cystitis, e.g., urinating about 11 times per night at about 1 month following the removal of the stent. Oral administration of CGP was again reported to be effective in reducing symptoms of hemorrhagic cystitis, e.g., the frequency of urination was reduced to about 7 times or about 2.5 times per night after oral administration of CGP for about 4 months or about 9 months, respectively, following the removal of the stent.

While not wishing to be bound by theory, these observations in a clinical setting suggest that a glycerophosphate salt can be used to prevent or treat a syndrome or a condition of discomfort associated with hemorrhagic cystitis, at least by palliating, or alleviating the syndrome or the condition of discomfort of the patient.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method of preventing or treating a syndrome or a condition of discomfort associated with hemorrhagic cystitis in a subject comprising administering to the subject an effective amount of a glycerophosphate salt, to interdict, prevent, palliate, alleviate, or delay the onset of the syndrome or the condition of discomfort of the subject associated with the hemorrhagic cystitis.

2. The method of claim 1, wherein the hemorrhagic cystitis is caused by a chemotherapy.

3. The method of claim 1, wherein the hemorrhagic cystitis is caused by a radiation therapy.

4. The method of claim 3, wherein the radiation therapy is a brachytherapy.

5. The method of claim 3, wherein the radiation therapy is a pelvic radiation therapy.

6. The method of claim 5, wherein the pelvic radiation therapy is used to treat an endometrial tumor, a cervical tumor, a rectal tumor, a prostate tumor, a genital tumor, a bone or soft tissue tumor.

7. The method of claim 6, wherein the genital tumor is a penile tumor or an ovarian tumor.

8. The method of claim 1, wherein the hemorrhagic cystitis is caused by a chemotherapy in combination with one or more additional therapies.

9. The method of claim 1, wherein the hemorrhagic cystitis is caused by a radiation therapy in combination with one or more additional therapies.

10. The method of claim 1, wherein the hemorrhagic cystitis is caused by viral infection.

11. The method of claim 1, wherein the syndrome or the condition of discomfort is pain.

12. The method of claim 1, wherein the glycerophosphate salt is selected from the group consisting of calcium glycerophosphate (CGP), magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, glycerophosphate strontium, and combinations thereof.

13. The method of claim 12, wherein the glycerophosphate salt is calcium glycerophosphate.

14. The method of claim 1, wherein the subject is a human subject or a non-human mammal.

15. The method of claim 1, wherein the glycerophosphate salt is administered to the subject by oral administration, topical administration, injection, infusion, or intravesical instillation.

16. The method of claim 15, wherein the glycerophosphate salt is administered topically to the bladder or the pelvic cavity of the subject.

17. The method of claim 1, wherein the glycerophosphate salt is administered to the subject in a composition of a form selected from the group consisting of a cream, an ointment, a gel, a tablet, a capsule, a pill, a caplet, a granule, a powder, a syrup, a solution, a syrup, an elixir, an emulsion, a suppository, and a suspension.

18. The method according to claim 1, wherein the effective amount of the glycerophosphate salt is about 0.1 gram to about 3.0 grams per administration.

19. The method of claim 1, wherein the glycerophosphate salt is administered to the subject prior to, together with, or after administering a chemotherapy to the subject.

20. The method of claim 1, wherein the glycerophosphate salt is administered to the subject prior to, together with, or after administering a radiation therapy to the subject.

21. The method of claim 1, wherein the glycerophosphate salt is administered to the subject prior to, together with, or after a bone marrow transplant.

22. A method of preventing or treating a syndrome or a condition of discomfort associated with a damage to a cell or a tissue of the bladder resulting from a radiation therapy or chemotherapy in a subject, comprising administering to the subject an effective amount of a glycerophosphate salt, to interdict, prevent, palliate, or alleviate the syndrome or the condition of discomfort of the subject associated with the damage.

23. The method of claim 22, wherein the syndrome or the condition of discomfort is mucositis in the bladder or the pelvic cavity of the subject.
